(19) United States
(12) Patent Application Publication
(10) Pub. No.: US 2010/0030329 A1
(43) Pub. Date: Feb. 4, 2010

(54) Annuloplasty Prosthesis

(76) Inventor: Robert William Mayo Frater, Bronxville, NY (US)

Correspondence Address: LADAS & PARRY LLP, 26 WEST 61ST STREET, NEW YORK, NY, 10023, US

(21) Appl. No.: 12/086,541

(22) PCT Filed: Dec. 18, 2006
(86) PCT No.: PCT/IB2006/054920

§ 371 Date: Apr. 15, 2009

Related U.S. Application Data
(60) Provisional application No. 60/751,746, filed on Dec. 19, 2005.

(51) Int. Cl. A61F 2/24

(52) U.S. Cl. 623/2.36

(57) ABSTRACT

An annuloplasty prosthesis includes a bowed core (10) having an inner edge (10) and an outer edge (12). A plurality of openings (16) are defined by, and spaced apart along, the core so that struts (18) are defined between adjacent openings (16). The openings and struts provide anchoring sites (20) for annuloplasty sutures in use anchoring the prosthesis to a heart annulus. The anchoring sites (20) are not adjacent the outer core edge (14).

THIS INVENTION relates to an annuloplasty prosthesis. It also relates to an annuloplasty kit.

According to one aspect of the invention, there is provided an annuloplasty prosthesis which includes
a bowed core having an inner edge and an outer edge; and
a plurality of openings defined by, and spaced apart along, the core so that struts are defined between adjacent openings, the openings and struts providing anchoring sites for annuloplasty sutures in use anchoring the prosthesis to a heart annulus, with the anchoring sites not being adjacent the outer core edge.

In use, the prosthesis can be anchored by means of sutures passing through the anchoring sites, to a heart annulus, with intrusion of the prosthesis into a flow passage defined by the heart annulus, being inhibited.

The bowed core may be generally C-shaped or D-shaped, when seen in plan view.

In one embodiment of the invention, the anchoring sites may be located about halfway between the inner core edge and the outer core edge. However, in another embodiment of the invention, the anchoring sites may be located closer to the inner core edge than to the outer core edge. In general, it is preferred that the openings, and hence the anchoring sites, be close to the inner core edge, or as close to the inner core edge as possible.

At least some of the openings may have an inner peripheral zone and an outer peripheral zone, the inner peripheral zone being located closer to the inner core edge while the outer peripheral zone is located closer to the outer core edge. The distances of the inner peripheral zones from the inner core edge is then not greater than the distances of the outer peripheral zones from the outer core edge. More preferably, the distances of the inner peripheral zones from the inner core edge may be less than the distances of the outer peripheral zones from the outer core edge.

The strut-to-strut dimension of at least some of the openings may be at least equal to the widths of adjacent struts. In some embodiments, the strut-to-strut dimension of at least some of the openings may be greater than the widths of adjacent struts.

The core may have a flattened cross-sectional profile, with its widest dimension extending between its inner edge and its outer edge.

The prosthesis may include a biocompatible cover covering the core. The biocompatible cover may be of biological tissue, e.g. a xenograft pericardium biological tissue.

The biocompatible cover may be secured to the core by means of aligning stitches which pass through at least some of the openings.

The biocompatible cover may include anchoring site markings which aid in identifying or locating the anchoring sites. The anchoring site markings may be in the form of marking stitches indicating the location of the struts.

According to a further aspect of the invention, there is provided an annuloplasty kit which includes:
at least one annuloplasty prosthesis as hereinbefore defined; and
auxiliary equipment facilitating use of the annuloplasty prosthesis during an annuloplasty procedure.

The kit may include a plurality of the annuloplasty prostheses of differing sizes.

The auxiliary equipment may include a bending tool with which to adjust the shape of the annuloplasty prosthesis.

The bending tool may be an out-of-plane bending tool which includes a pair of spaced prongs each defining a holding formation shaped and dimensioned to hold a portion of the annuloplasty prosthesis, and a shaped press, operative in use to exert pressure on a portion of the annuloplasty prosthesis located between the portions of the prosthesis held in the holding formations.

The out-of-plane bending tool may include a first member defining the spaced prongs, and a second member pivotally mounted or mountable to the first member, with the shaped press being defined by or receivable on the second member, so that pressure is in use exerted by pivotal displacement of the second member relative to the first member.

Instead, the bending tool may be an in-plane bending tool which includes a bending jig defining a slot matching the curvature of the annuloplasty prosthesis in which a portion of the prosthesis is receivable, and the bending jig serving to limit out of plane bending, and a force applier with which to exert pressure on the prosthesis.

Both an out-of-plane bending tool and an in-plane bending tool may be provided.

The auxiliary equipment may instead, or additionally include a suture guide for aiding in placing sutures in a heart annulus at locations that correspond to the locations of the anchoring sites of the annuloplasty prosthesis.

The suture guide may be in the form of a guide body, with a perimeter of the guide body defining notches that correspond to the locations of the anchoring sites of the annuloplasty prosthesis.

Alternatively the suture guide may be in the form of a guide body, the body defining radially extending projections having ends indicating the locations of the anchoring sites of the annuloplasty prosthesis.

Regardless of the form of the suture guide, it may include securing formations with which temporarily to secure the suture guide to a heart annulus.

The kit may include a plurality of the suture guides, with the suture guides being of differing sizes to match the sizes of the prostheses.

The auxiliary equipment may instead, or additionally, include sizers to aid a surgeon in selecting a correctly sized prosthesis.

Each sizer may be in the form of a sizing body, a perimeter of the body matching a curve connecting the anchoring sites of a particularly sized prosthesis.

The auxiliary equipment may instead, or additionally, include a holding device with which to hold the prosthesis in position while anchoring the prosthesis to a heart annulus. The holding device may include a generally C-shaped holder having an end face along an inner edge of the holder, the end face defining a slot extending along its length, with the holder being shaped and dimensioned such that when an outer portion of the prosthesis is held within the slot, the anchoring sites of the prosthesis are exposed.

The holding device may include an additional elongate holding component spanning the gap between the free ends of the bowed end face of the C-shaped holder, the elongate holding component having a face defining a slot adapted to accommodate the straight portion of a D-shaped prosthesis when the bowed portion thereof is accommodated in the slot of the holder, so that anchoring sites along the straight portion of the D-shaped prosthesis are exposed. The additional elongate holding component may thus be releasably attached to the holder.

According to yet a further aspect of the invention, there is provided a method of performing an annuloplasty procedure, the method including anchoring an annuloplasty prosthesis as hereinbefore described to a heart annulus by means of annuloplasty sutures which pass through the anchoring sites, thereby inhibiting intrusion of the prosthesis into a blood flow passage defined by the heart annulus.

Further features of the invention will become apparent from the following description presented by way of example with reference to the accompanying diagrammatic drawings.

In the drawings:

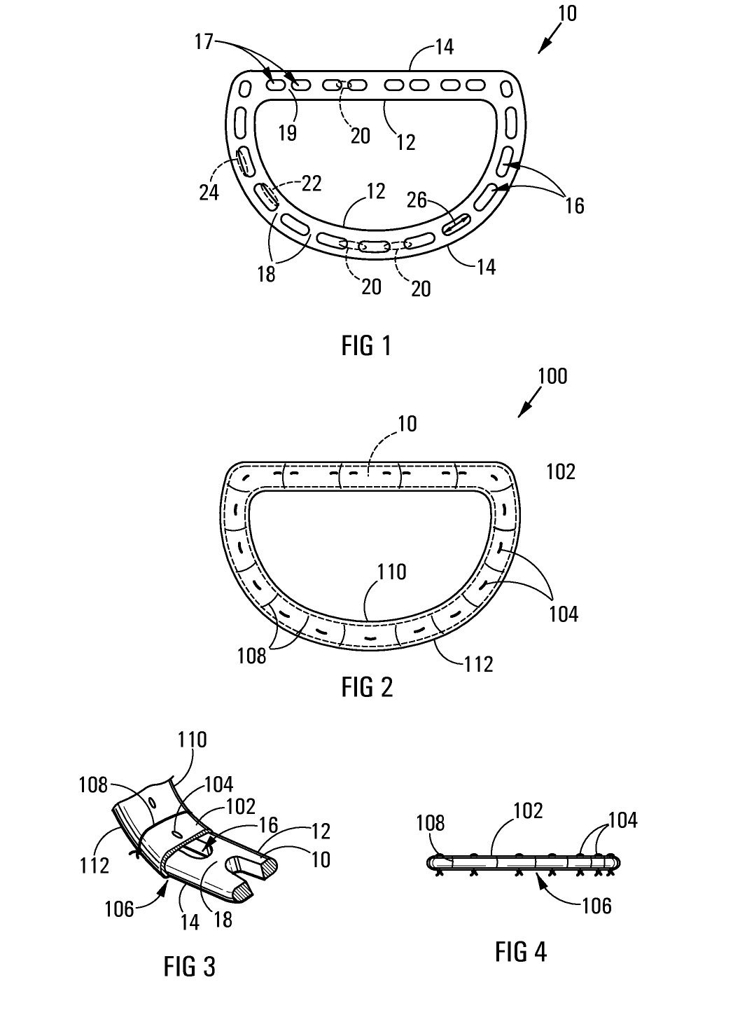

FIG. 1 shows a plan view of a core of an annuloplasty prosthesis according to a first embodiment of the invention;

FIG. 2 shows a plan view of a prosthesis incorporating the core of FIG. 1;

FIG. 3 shows a three-dimensional view of a portion of the prosthesis of FIG. 2, with a portion of its biocompatible cover removed;

FIG. 4 shows a side elevation of the prosthesis of FIG. 2;

FIG. 5 shows a plan view of a core of a prosthesis according to a second embodiment of the invention;

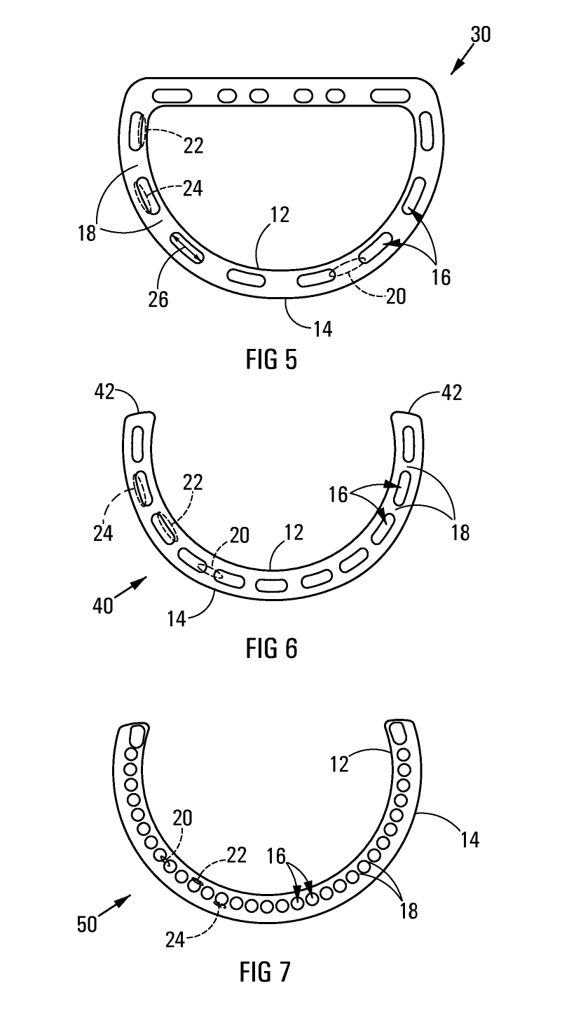

FIG. 6 shows a plan view of a core of a prosthesis according to a third embodiment of the invention;

FIG. 7 shows a plan view of a core of a prosthesis according to a fourth embodiment of the invention;

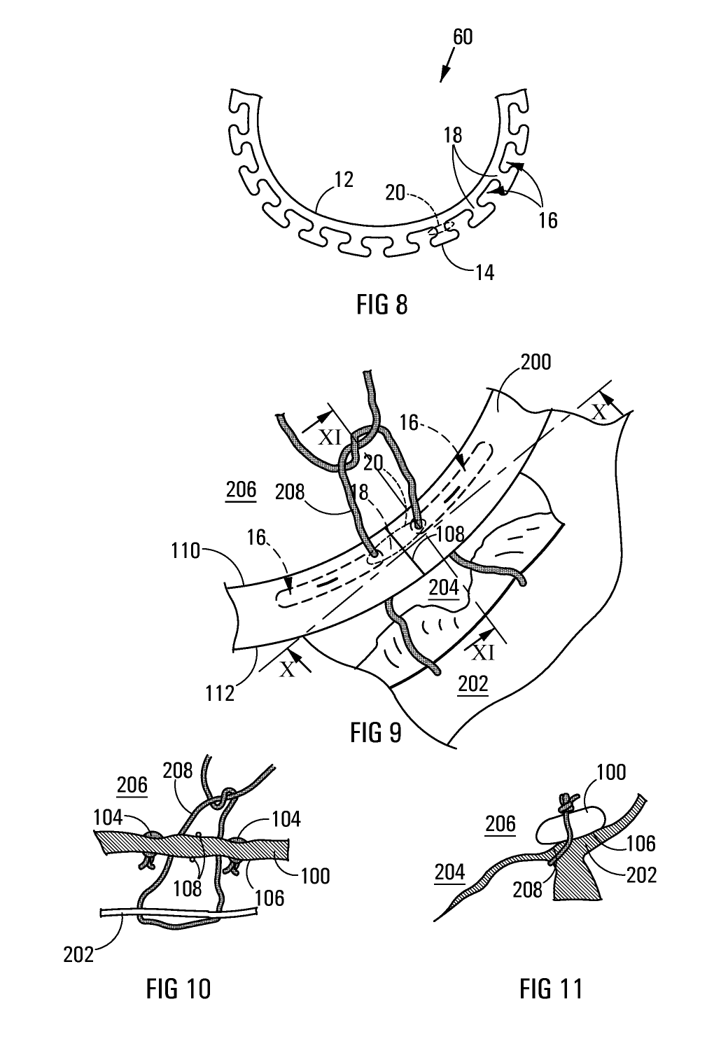

FIG. 8 shows a plan view of a core of a prosthesis according to a fifth embodiment of the invention still;

FIG. 9 shows a three-dimensional view of a portion of a prosthesis according to the invention being secured to a heart annulus;

FIG. 10 is a section taken along line X-X in FIG. 9;

FIG. 11 is a section taken along line XI-XI in FIG. 9, showing the prosthesis secured to the heart annulus;

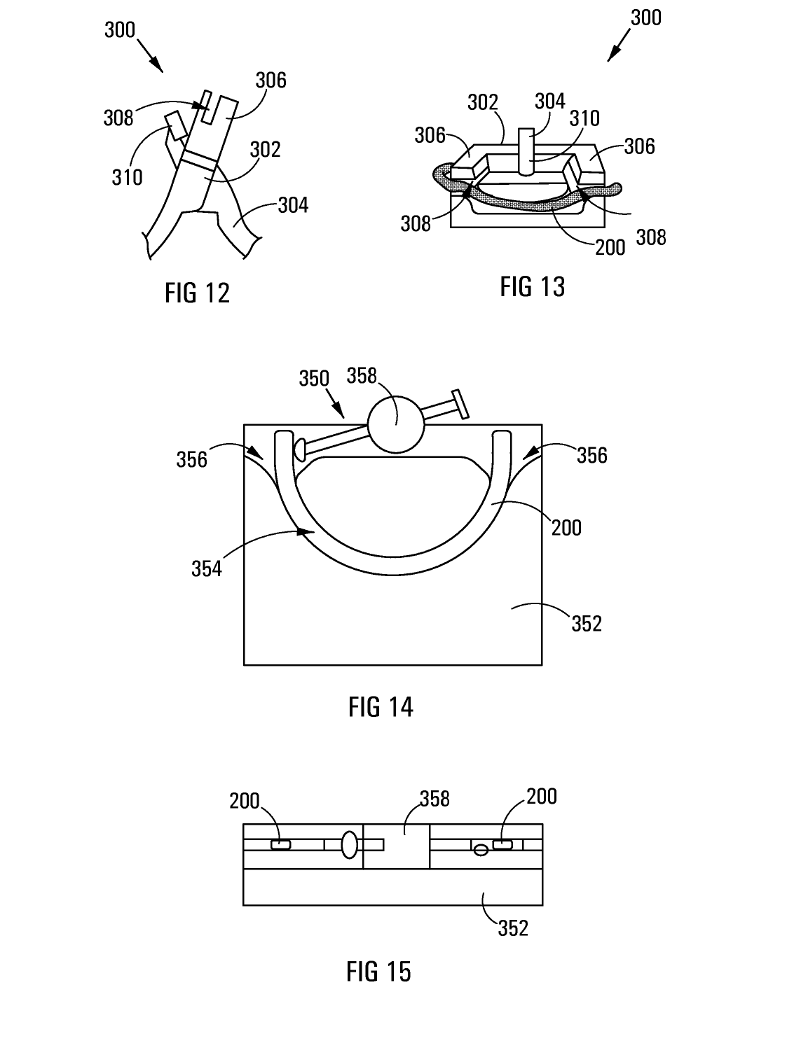

FIG. 12 shows a side view of an out-of-plane bending tool forming part of an annuloplasty kit according to the invention;

FIG. 13 shows an end view of the out-of-plane bending tool of FIG. 12, with a prosthesis held by the bending tool;

FIG. 14 shows a plan view of an in-plane bending tool forming part of an annuloplasty kit according to the invention;

FIG. 15 shows a side elevation of the in-plane bending tool of FIG. 14;

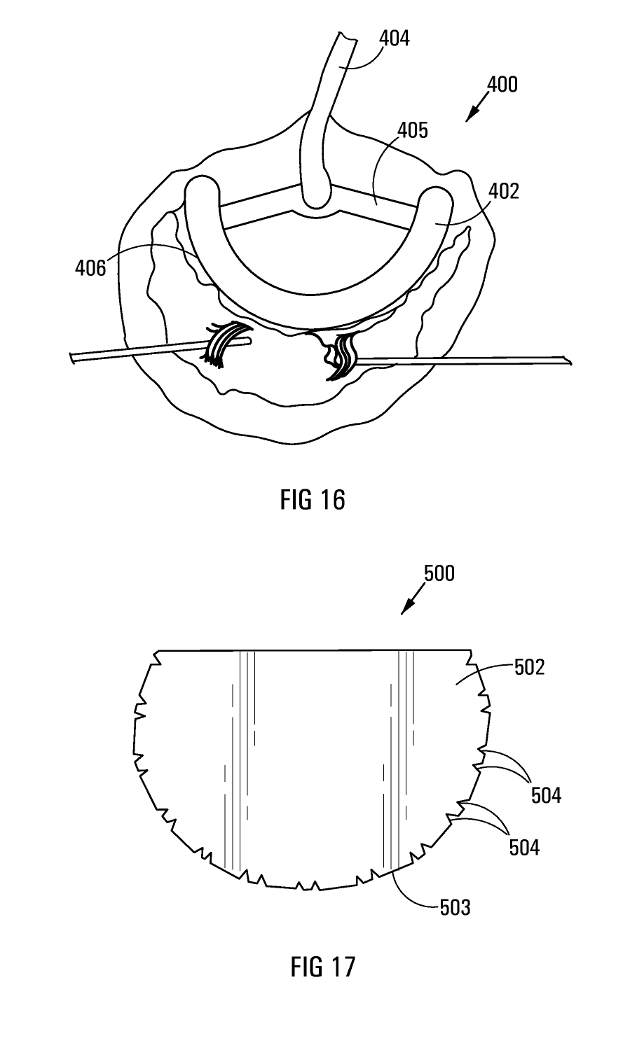

FIG. 16 shows a plan view of a sizer forming part of an annuloplasty kit according to the invention;

FIG. 17 shows a plan view of one embodiment of a suture guide forming part of an annuloplasty kit according to the invention;

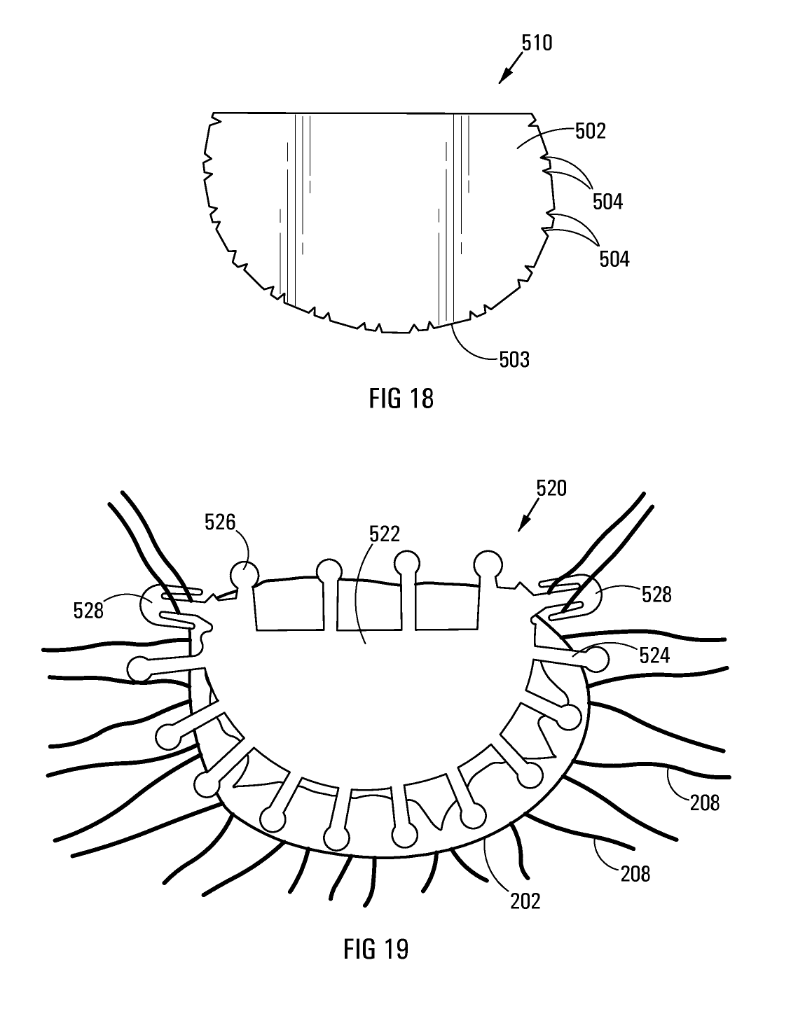

FIG. 18 shows a plan view of a different embodiment of a suture guide forming part of an annuloplasty kit according to the invention;

FIG. 19 shows a plan view of yet a different embodiment of a suture guide forming part of an annuloplasty kit according to the invention, in use;

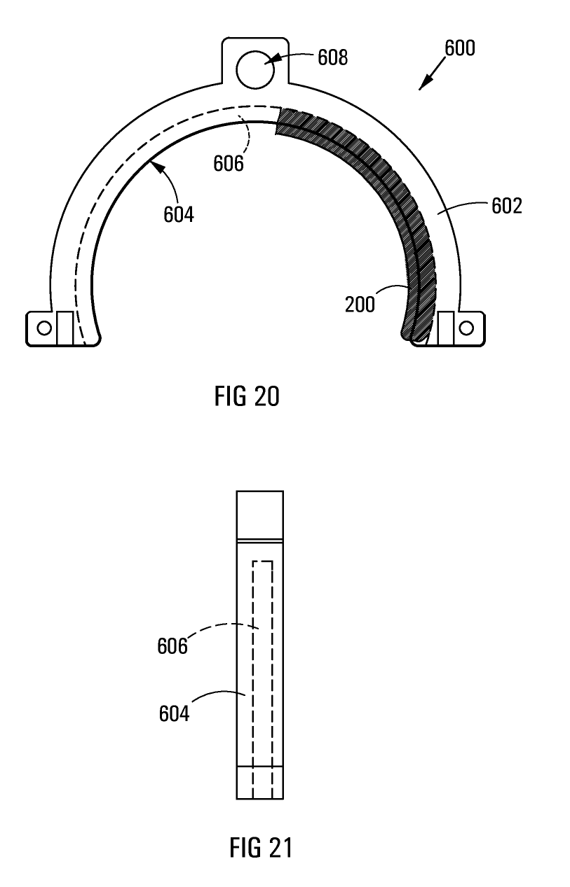

FIG. 20 shows a plan view of a holding device forming part of an annuloplasty kit according to the invention; and

FIG. 21 shows a side elevation of the holding device of FIG. 20.

With reference to FIG. 1, reference numeral 10 generally indicates a core of an annuloplasty prosthesis in accordance with a first embodiment of the invention. The core 10 is bowed, being generally D-shaped when seen in plan view, and has an inner edge 12 and an outer edge 14. A plurality of openings 16 is defined by, and spaced along, the curved portion of core 10 with struts 18 being defined between adjacent openings 16. The openings 16 pass through a plane defined between the inner core edge 12 and the outer core edge 14. The openings 16 and struts 18 provide anchoring sites 20 for annuloplasty sutures in use securing the annuloplasty prosthesis to a heart annulus, with each annuloplasty suture then passing through adjacent openings 16 and crossing over the strut 18 between the adjacent openings 16. The anchoring sites 20 are not adjacent the outer core edge 14; rather, the anchoring sites 20 are located about halfway between the inner core edge 12 and the outer core edge 14.

Openings 17 are provided in the straight portion of the core 10, with struts 19, being defined between adjacent openings 17. It will be appreciated that the openings 17 and struts 19 also provide anchoring sites 20 as hereinbefore described.

It will also be apparent from FIG. 1 that the openings 16 each have an inner peripheral zone 22 which is closer to the inner core edge 12, and an outer peripheral zone 24 which is closer to the outer core edge 14. For the core 10, the distances that the inner peripheral zones 22 are from the inner core edge 12 is not greater than the distances that the outer peripheral zones 24 are from the outer core edge 14.

For the core 10, the strut-to-strut dimensions 26 of the openings 16 are greater than the widths of the adjacent struts 18.

As best shown in FIG. 3 and FIG. 4, the core 10 has a flattened cross-sectional profile, with its widest dimension extending between its inner edge 12 and its outer edge 14. The core 10 is constructed from a material having an appropriate degree of stiffness in thin cross-sections to resist systolic forces, typically being in the range of 6 to 8 Newtons, while still being sufficiently pliable to allow for adjustment of its shape by a surgeon while performing an annuloplasty procedure. The material should also be biocompatible. Typically, the core 10 is of titanium.

Referring now to FIG. 2, reference numeral 100 generally indicates an annuloplasty prosthesis incorporating the core 10. The prosthesis 100 includes a biocompatible cover 102 covering the core 10. The cover 102 is typically a xenograft pericardium biologic tissue which has been glutaraldehyde tanned and subjected to an anticalcifying detoxifying treatment. In the Inventor's experience, this type of biologic tissue has given good results when used as a sewing ring for artificial heart valves, and features substantially quicker and more complete and uniform healing to it by a host than synthetic cloths. In addition, the Inventor has experienced that this type of cover is less thrombogenic than synthetic cloth coverings and in all likelihood will not result in hemolysis when struck by a jet of insufficiency. It has been experimentally demonstrated that xenograft pericardium subjected to the treatment in the manner described above shows spontaneous growth on it of host derived endothelial cells. It will be appreciated that other biocompatible or biologic tissue materials, preferably having the favourable properties of not causing or encouraging thrombosis, hemolysis, and immune or inflammatory responses, may be used as alternatives for the cover 102.

The cover 102 is secured to the core 10 by means of aligning stitches 104 passing through the openings 16. As best shown in FIG. 4, the knots of the aligning stitches 104 are all located on a first flat side 106 of the prosthesis 100.

The biocompatible cover 102 naturally obscures the location of the anchoring sites 20. In order to aid a surgeon, the biocompatible cover 102 includes anchoring site markings in the form of marking stitches 108 which aid in identifying or locating the anchoring sites 20. As best shown in FIG. 3, each marking stitch 108 encircles the biocompatible cover 102 at the position of a strut 108, thereby indicating the location of the struts 18 and hence the anchoring sites 20. Each marking stitch 108 is attached to the biocompatible cover 102 at an inner perimeter 110 and an outer perimeter 112 of the prosthesis 100, thereby to maintain the position of the marking stitch 108. It will be appreciated that different types of anchoring site markings are possible, e.g. markings made using an indelible non-toxic dye. It will also be appreciated that the aligning stitches 104 also act as guides in locating the openings 16.

As best shown in FIG. 3, the prosthesis 100, as a result of the flattened cross-sectional profile of the core 10, has a flattened cross-sectional profile with its widest dimension extending between the inner perimeter 110 and the outer perimeter 112. The anchoring sites 20 are thus also located about halfway between the inner perimeter 110 and the outer perimeter 112.

FIGS. 5 to 8 each show a core of an annuloplasty device according to different embodiments of the invention. These cores have many features in common with the core 10, and such features are indicated by identical reference numerals.

Referring to FIG. 5, reference numeral 30 generally indicates a core which is generally D-shaped when viewed in plan. In the core 30, the anchoring sites 20 are located closer to the inner core edge 12 than to the outer core edge 14. It is also apparent from FIG. 5 that, for the core 30, the distances of the inner peripheral zones 22 from the inner core edge 12 are less than the distances of the outer peripheral zones 24 from the outer core edge 14.

With reference to FIG. 6, reference numeral 40 generally indicates a core which is generally C-shaped when viewed in plan. The core 40 is similar to the core 10, but, since the straight portion of the D-shape is not present, it features two free trigonal ends 42. The positions of the anchoring sites 20 relative to the inner core edge 12 and the outer core edge 14, as well as the relative distances of the inner peripheral zones 22 and outer peripheral zones 24 from the inner core edge 12 and the outer core edge 14 respectively are as were described for the core 10.

Referring now to FIG. 7, reference numeral 50 generally indicates a core which is generally C-shaped when viewed in plan. The openings 16 of the core 50 are circular. The positions of the anchoring sites 20 relative to the inner core edge 12 and the outer core edge 14, as well as the relative distances of the inner peripheral zones 22 and outer peripheral zones 24 from the inner core edge 12 and the outer core edge 14 respectively are similar to those described for the core 30.

Although not shown, the cores 30, 40, 50 all feature a flattened cross-sectional profile similar to that of the core 10 and similar considerations regarding the stiffness of the cores 30, 40, 50 apply as discussed above in connection with the core 10. The cores 10, 30, 40, 50 are thus all rigid cores, with the cores 10, 30 being D-shaped rigid cores, and the cores 40, 50 being C-shaped rigid cores.

Situations may arise where it is preferable to use a semi-rigid annuloplasty prosthesis, i.e. having the feature that it resiliently responds to systolic forces. With reference to FIG. 8, reference numeral 60 generally indicates a semi-rigid core in accordance with an embodiment of the invention. The core 60 is generally C-shaped when viewed in plan, and has the feature that the openings 16 open out on to the outer edge 14, i.e. the openings 16 interrupt the outer edge 14. The core 60 is thus semi-rigid and capable of resiliently responding to systolic forces. Note that the core openings 16 and struts 18 still provide the anchoring sites 20 which are, for the embodiment shown, located about halfway between the inner core edge 12 and the outer core edge 14.

The cores 30, 40, 50, 60 are incorporated in annuloplasty prostheses as discussed above in respect of the core 10, i.e. they are covered with a biocompatible cover 102, to form annuloplasty prostheses that are similar to the prosthesis 100, for convenience hereinafter referred to generally by reference numeral 200. It will be appreciated that by selecting an appropriate core, it is possible to form rigid D-shaped annuloplasty prosthesis (core 10 or core 30), rigid C-shaped annuloplasty prostheses (core 40 or core 50) or a semi-rigid C-shaped annuloplasty prostheses (core 60).

In use, the prosthesis 200 is used when performing an annuloplasty procedure, i.e. to effect mitral valve repairs rectifying dominant mitral valve insufficiency. This typically involves permanently narrowing and shaping the mitral valve annulus. As shown in FIG. 9, the prosthesis 200 is anchored or secured to a heart annulus 202 defining a mitral valve flow passage 204. The heart annulus 202 is at the junction of the valve leaflets and the entrance to the ventricle. Similar to known annuloplasty prostheses, the prosthesis 200 is oriented such that, in the case of a D-shaped prosthesis, its straight portion, and in the case of a C-shaped prosthesis, its gap, is nearest the anterior of the heart, while the bowed portion is nearest the posterior of the heart. As best shown in FIG. 10, the prosthesis 200 is oriented such that its first flat side 106 faces the annulus 202, while its opposite flat side faces the atrium 206 of the heart, so that the knots of the aligning sutures 104 are sandwiched between the prosthesis 200 and the annulus 202.

Each anchoring site 20 is anchored or secured to the annulus 202 by means of an annuloplasty suture 208, typically being a suture of the mattress type. The aligning stitches 104 and the marking stitches 108 assist the surgeon in placing the annuloplasty sutures 208 in the prosthesis 200. The arms of each annuloplasty suture 208 pass through adjacent openings 16 and cross over the strut 18 between the adjacent openings. It is important to note that the prosthesis 200 is thus anchored to the annulus 202 by effectively anchoring the rigid or semi-rigid core 10, 30, 40, 50, 60 of the prosthesis 200 to the annulus 202, thus ensuring firm contact between the prosthesis 200 and the annulus 202.

Advantageously, the Inventor believes that, by using the prosthesis 100, 200 in accordance with the invention, it is thus possible for a surgeon to make a fair prediction of the post-operative shape and size of the annulus 202. Furthermore, as best shown in FIG. 11, the location of the anchoring sites 20 and openings 16 relative to the inner core edge 12 and the outer core edge 14, and thus also the inner perimeter 110 and the outer perimeter 112 of the prosthesis 200, means that intrusion of the prosthesis 200 into the flow passage 204 is inhibited or minimized. The Inventor believes that this feature, together with the flat cross-sectional profile of the prosthesis 200, advantageously limits interference with blood flow along the flow passage 204 and hence also the probability that the prosthesis 200 will be contacted by a jet of residual mitral valve insufficiency. In the unlikely event that such contact does take place, the Inventor believes that the biologic cover 102, as explained above, limits the impact of such contact. Also, the Inventor believes that the inhibited protrusion of the prosthesis 200 into the flow passage 204 reduces risk associated with abnormal flow patterns that may lead to platelet activation. The use of the prosthesis 200 also results in broad prosthesis surface contact with annulus tissue, which will promote tissue ingrowth into, or integration with, the biologic cover 102.

In order to realise the full potential of the prosthesis 200 as hereinbefore described, it is preferred to employ auxiliary equipment especially adapted for use with the prosthesis 200. The prosthesis 200 may thus form part of an annuloplasty kit including the prosthesis 200 as well as auxiliary equipment. Typically, such a kit will include a number of the prostheses 200 of varying sizes and types, with a surgeon selecting a size and type suited to a particular patient anatomy and diseased condition.

A skilled person knows that there is endless variability in normal and abnormal anatomy, and, as such, no single ideal shape of annuloplasty prosthesis exists that is suited for all annuloplasties. As already discussed above, the material of construction of the core 10, 30, 40, 50, 60 of the prosthesis 200 according to the invention is selected to have an appropriate degree of stiffness in thin cross-sections to resist systolic forces, while still being sufficiently pliable to allow for adjustment of its shape by a surgeon while performing annuloplasty procedures. Advantageously, it is thus possible for a surgeon permanently to adjust the shape of the prosthesis 200 to better match the patient anatomy. The shape of the prosthesis 200 after adjustment thus continues to be unaffected by systolic forces. It is important to note that adjustment of the prosthesis 200 while performing an annuloplasty procedure, i.e. at the operating table, is preferred. Advantageously, a fair match to the patient anatomy is possible when using a kit having far fewer sizes and types of prostheses 200 than would be the case if such permanent adjustment of the shape of the prostheses 200 at the operating table was not possible.

The auxiliary equipment making up the annuloplasty kit thus typically includes bending tools with which to adjust or vary the shape of the prosthesis 200. In general, such bending tools include components securing portions of the prosthesis 200 not to be bent, and a space adjacent to the secured portions allowing bending to take place. The bending tools are typically hand-held.

With reference to FIGS. 12 and 13, reference numeral 300 generally indicates an out-of-plane bending tool forming part of an annuloplasty kit in accordance with the invention. The bending tool 300 includes a first member 302 and a second member 304 pivotally mounted to the first member. An end of the first member 302 defines a pair of spaced prongs 306, with each prong 306 defining a groove 308 along its end face, the grooves 308 being aligned with each other. Each of the grooves 308 is shaped and dimensioned to hold a portion of the prosthesis 200, and is thus a holding formation. An end of the second member 304 defines a shaped press 310. The members 302, 304 are configured such that the shaped press 310 is pivotally displaceable through a space defined between the prongs 306.

In use, the bending tool 300 is used to effect out-of-plane bending of the prosthesis 200, i.e. to bend a portion of the prosthesis 200 out of the plane of the curvature of the prosthesis 200. Portions of the prosthesis 200 on either side of the portion to be bent are received and held in the holding formations formed by the grooves 308, with the portion to be bent located between the holding formations. The shaped press 310 is then used to exert pressure on the portion to be bent by pivotal displacement of the second member 304 relative to the first member 302.

If desired, different degrees and shapes of bending may be achieved by using bending tools of different dimensions and shapes similar to the bending tool 300 as described above.

The auxiliary equipment typically also includes an in-plane bending tool. With reference to FIGS. 14 and 15, reference numeral 350 generally indicates an in-plane bending tool forming part of an annuloplasty kit in accordance with the invention. The bending tool 350 includes a bending jig 352 having formations defining a slot 354 matching the curvature of the prosthesis 200 in which a portion of the prosthesis 200 is receivable. The slot 354 has splayed ends 356 to facilitate bending. Typically, about two thirds of a C-shaped prosthesis is receivable in the slot 354. The bending tool 350 further includes a force applier 358 with which to exert pressure on the prosthesis 200.

In use, a portion of the prosthesis is received and held in the slot 354. The jig 352 thus serves to limit out-of-plane bending of at least the portion of the prosthesis 200 held in the slot 354. The force applier 358 is then used to exert pressure on a portion of the prosthesis 200 in a direction generally aligned with the plane of curvature of the prosthesis 200. Typically, the bending tool 350 is used with C-shaped prostheses to bend trigonal ends of the prosthesis 200 (corresponding to the trigonal ends 42 of the core 40, 50, 60) outwardly.

It is thus possible for a surgeon to adjust the shape of the prosthesis 200 to better match the patient anatomy in a controlled fashion using the bending tools 300, 350. One particular example where such adjustment is appropriate is using the bending tool 350 to widen an intertrigonal distance between the trigonal ends 42 of the prostheses 200 when it is a C-shaped prosthesis, thereby to compensate for enlargement of the anterior leaflet of the mitral valve, a condition associated with chronic ventricular dilation and some myopathy cases. It will be appreciated that, in such cases, selecting an unadjusted prosthesis 200 matching the enlarged anterior leaflet, will typically result in having the bowed portion of the prosthesis 200 well outside the annulus 202. A further example is found in cases where the normal systolic saddle shape of the annulus 202 has been lost, e.g. in cases of myocordial ischemic disease or cardiomyopathy. The saddle shape may then be restored by using the bending tool 300 to effect an appropriate out out-of-plane bend in the bowed portion of the prosthesis 200, near either the trigonal ends 42 (in case the prosthesis 200 is C-shaped), or the straight portion (in case the prosthesis 200 is D-shaped). In cases where the prosthesis 200 is D-shaped, yet a further example is to bend the straight portion of the prosthesis 200 out-of-plane and operatively towards the atrium 206 using the tool 300, thereby in use to move the base of the anterior leaflet towards the posterior leaflet and so improve coaptation and avoid systolic anterior movement.

The auxiliary equipment typically also includes sizers of various sizes to aid a surgeon in selecting a correctly sized prosthesis 200. Referring now to FIG. 16, reference numeral 400 generally indicates a sizer forming part of the annuloplasty kit in accordance with the invention. The sizer 400 includes a sizing body in the form of a planar bowed band 402 and a handle 404 attached to the sizing body 402 by means of angled struts 405. An outer peripheral edge 406 of the sizing body 402 defines a curve matching a curve connecting the anchoring sites 20 of the bowed portion of the prosthesis 200. It will thus be apparent that the annuloplasty kit in accordance with the invention typically includes a number of sizers 400 of varying sizes to match the various sizes of the prostheses 200 forming part of the kit. The same sizer may be used for C-shaped and D-shaped rings. In some embodiments the sizer 400 may be of a translucent material. Preferably the sizer 400 is steam sterilizable.

In use, the sizer 400 aids a surgeon in selecting the prosthesis 200 of an appropriate size. The sizer is designed to be matched to an anterior leaflet of the heart, the anterior leaflet having been flattened out by traction of the main heart chordae. The spaces or gaps in and around the sizer 400 make it possible to see the anterior leaflet while the sizer 400 is in use. Since the outer perimeter 406 matches the location of the anchoring sites 20 of a matching prosthesis 200, and because, as explained above, there is a close post-operative match between the state of the annulus 202 and the location of the anchoring sites 20 it is possible for a surgeon to make a fair prediction of the post-operative state of the annulus 202 using the sizer 400.

When performing annuloplasty procedures, it is preferable first to place the annuloplasty sutures 208 in the annulus 202 prior to placing and anchoring the prosthesis 200 to the annulus. Accordingly, the auxiliary equipment further include suture guides, typically of various sizes, to aid a surgeon in placing the annuloplasty sutures 208 prior to placing and anchoring the prosthesis 200 to the annulus 202. Referring now to FIG. 17, reference numeral 500 generally refers to a suture guide forming part of an annuloplasty kit in accordance with the invention. The suture guide 500 includes a planar guide body 502 having a form similar to the annulus 202 in dilated form. Notches 504 on the curved perimeter of the guide body 502 indicates sites for the placement of annuloplasty sutures 208 in the annulus 202 that will then match the location of the anchoring sites 20 of a corresponding prosthesis 200. It is apparent that the notches 504 are arranged to match the prosthesis 200 incorporating cores of the type of the cores 10, 30, 40 and 60. The curved perimeter 503 spans 216° in total and is made up of 12 arcs of 18° each, with an end of each arc corresponding to the position of the anchoring site 20 of the matching prosthesis 200. A pair of the notches 504 straddles the end of each arc, the pair thus indicating the locations of the arms of an anchoring suture 208.

In use, the suture guide 500 is chosen to match the annulus 202 in dilated form. The curved perimeter 503 is held close to the annulus 202 tissue with the notches 504 indicating where annuloplasty sutures 208 are to be placed. The Inventor believes that such guided placement of the annuloplasty sutures 208 produces an undistorted and even reduction and narrowing of the annulus 202.

It is conceivable that patient anatomy may feature an eccentrically dilated annulus. For example, an annulus that is eccentrically dilated on the right side is a typical consequence of an inferior infarct. As such, the auxiliary equipment may also include eccentric suture guides. Referring now to FIG. 18, reference numeral 510 generally indicates an eccentric suture guide. Apart from its eccentricity, the suture guide 510 is identical to the suture guide 500 and identical reference numerals are used. Similarly to the suture guide 500, the notches 504 of the suture guide 510 are also arranged in pairs with each pair being at an end of an arc of 180. Consequently, the notches 504 are spaced wider apart along the eccentric portion of the perimeter 503.

The suture guide 510 is used similarly to the suture guide 500. Advantageously, the wider spacing of the notches 504 along the eccentric portion of the perimeter 503 automatically results in a greater degree of narrowing of the eccentrically dilated part of the annulus when the prosthesis 200 is anchored to the annulus 202.

Another form of suture guide, generally indicated by reference numeral 520 is shown in FIG. 19. The suture guide 520 also forms part of an annuloplasty kit in accordance with the invention. The suture guide 520 includes a planar guide body 522 having a D-shaped outline, and a plurality of radially extending projections 524 along its curved portion and a plurality of projections 526 along its straight portion. The suture guide also includes two securing formations 528 extending outwardly where the straight and curved portions of the guide body 522 meet. In the embodiment shown, the projections 524 are arcuately spaced apart by 18°, i.e. similarly to the pairs of notches 504 of the suture guides 500, 510. The projections 524 thus correspond to the location of the anchoring sites 20 along the bowed portion of the matching prosthesis 200, while the projections 526 correspond to the location of the anchoring sites 20 along the straight portion. In some embodiments, the projections 524, 526 may have a widened end portion, with the sides of the widened end portion giving a better indication of the width of the annuloplasty sutures.

The suture guide 520 is used similarly to the suture guides 500, 510, with the exception that the securing formations may be used to temporarily suture the suture guide 520 to the annulus 202. It will be apparent that such securing formations may also form part of the suture guides 500, 510. Advantageously, additional eccentric suture guides 520 are not required, since, as shown in FIG. 19, the length of the projections 524, 526 automatically accounts for eccentricity of the annulus 502.

The Inventor believes that combined use of the prosthesis 200, the sizer 400 and the suture guide 500, 510, 520 as hereinbefore described will result in a suitable number of correctly spaced annuloplasty sutures 208 being used in order to achieve desired narrowing and reduction of the annulus 202. The Inventor also believes that the above holds true even for most eccentrically dilated annuli 202.

The annuloplasty kit according to the invention may further include a holding device with which to hold the prosthesis 200 while it is being anchored to the annulus 202. With reference to FIGS. 20 and 21, there is shown such a holding device, generally indicated by reference numeral 600. The holding device 600 has a generally C-shaped holder 602 having an inner edge defining an edge face 604. A slot 606 extends along the length of the edge face 604. The holding device 600 also includes a handle (not shown) attachable to the holder 602 by means of a handle formation 608 defined by the holder 602. The holder 602 is selected to match the prosthesis 200, so that when an outer portion of the prosthesis 200 is received and held in the slot 606, the anchoring sites 20 of the prosthesis 200 are exposed.

In use, the prosthesis 200 is received in the slot 606 and thus conveniently held while the annuloplasty sutures 208 are completed so to anchor the prosthesis to the annulus 202.

To hold a D-shaped prosthesis, the holding device may include an additional elongate holding component (not shown) which is releasably attached to the holder 602 such that it spans the free ends of the slot 606. The elongate holding component will then have a face defining a slot adapted to accommodate the straight portion of the D-shaped prosthesis while the bowed portion thereof is accommodated in the slot 606 of the holder 602. It will be appreciated that the slot of the additional elongate holding component will then also be such that the anchoring sites along the straight portion of the D-shaped prosthesis are exposed. The elongate holding component is typically releasably attached to the holder 602, e.g. by suitable clips.

1. An annuloplasty prosthesis which includes
a bowed core having an inner edge and an outer edge, and having a flattened cross-sectional profile, with its widest dimension extending between its inner edge and its outer edge; and
a plurality of openings defined by, and spaced apart along, the core so that struts are defined between adjacent openings, the openings and struts providing anchoring sites for annuloplasty sutures in use anchoring the prosthesis to a heart annulus, with the anchoring sites not being adjacent the outer core edge and with at least some of the openings having an inner peripheral zone and an outer peripheral zone, the inner peripheral zone being located closer to the inner core edge while the outer peripheral zone is located closer to the outer core edge, with the distances of the inner peripheral zones from the inner core edge being less than the distances of the outer peripheral zones from the outer core edge.

2. The prosthesis as claimed in claim 1, in which the bowed core is generally C-shaped or D-shaped, when seen in plan view.

3. The prosthesis as claimed in claim 1, in which the anchoring sites are located closer to the inner core edge than to the outer core edge.

4. The prosthesis as claimed in claim 1, in which the strut-to-strut dimension of at least some of the openings is at least equal to the widths of adjacent struts.

5. The prosthesis as claimed in claim 4, in which the strut-to-strut dimension of at least some of the openings is greater than the widths of adjacent struts.

6. The prosthesis as claimed in claim 1, which includes a biocompatible cover covering the core.

7. The prosthesis as claimed in claim 6, in which the biocompatible cover is a xenograft pericardium biologic tissue.

8. The prosthesis as claimed in claim 6, in which the biocompatible cover is secured to the core by means of aligning stitches which pass through at least some of the openings.

9. The prosthesis as claimed in claim 6, in which the biocompatible cover includes anchoring site markings which aid in identifying or locating the anchoring sites.

10. The prosthesis as claimed in claim 9, in which the anchoring site markings are in the form of marking stitches indicating the location of the struts.

11. An annuloplasty kit which includes at least one annuloplasty prosthesis as claimed in claim 1, and
auxiliary equipment facilitating use of the annuloplasty prosthesis during an annuloplasty procedure.

12. The kit as claimed in claim 11, in which the auxiliary equipment includes a bending tool with which to adjust the shape of the annuloplasty prosthesis.

13. The kit as claimed in claim 12, wherein the bending tool is an out-of-plane bending tool which includes
a pair of spaced prongs each defining a holding formation shaped and dimensioned to hold a portion of the annuloplasty prosthesis; and
a shaped press, operative in use to exert pressure on a portion of the annuloplasty prosthesis located between the portions of the prosthesis held in the holding formations.

14. The kit as claimed in claim 13, in which the out-of-plane bending tool includes a first member defining the spaced prongs, and a second member pivotally mounted or mountable to the first member, with the shaped press being defined by or receivable on the second member, so that pressure is in use exerted by pivotal displacement of the second member relative to the first member.

15. The kit as claimed in claim 12, wherein the bending tool is an in-plane bending tool which includes
a bending jig defining a slot matching the curvature of the annuloplasty prosthesis in which a portion of the prosthesis is receivable, and the bending jig serving to limit out of plane bending; and
a force applier with which to exert pressure on the prosthesis.

16. The kit as claimed in claim 11, in which the auxiliary equipment includes a suture guide for aiding in placing sutures in a heart annulus at locations that correspond to the locations of the anchoring sites of the annuloplasty prosthesis.

17. The kit as claimed in claim 16, in which the suture guide is in the form of a guide body, with a perimeter of the guide body defining notches that correspond to the locations of the anchoring sites of the annuloplasty prosthesis.

18. The kit as claimed in claim 16, in which the suture guide is in the form of a guide body, the body defining radially extending projections having ends indicating the locations of the anchoring sites of the annuloplasty prosthesis.

19. The kit as claimed in claim 16, in which the suture guide includes securing formations with which temporarily to secure the suture guide to a heart annulus.

20. The kit as claimed in claim 11, in which the auxiliary equipment includes sizers to aid a surgeon in selecting a correctly sized prosthesis.

21. The kit as claimed in claim 20, in which the sizers are in the form of sizing bodies, a perimeter of each body matching a curve connecting the anchoring sites of the bioprosthesis.

22. The kit as claimed in claim 11, in which the auxiliary equipment includes a holding device with which to hold the prosthesis in position while anchoring the prosthesis to a heart annulus.

23. The kit as claimed in claim 22, in which the holding device includes a generally C-shaped holder having a bowed end face along an inner edge of the holder, the end face defining a slot extending along its length, with the holder being shaped and dimensioned such that when an outer portion of a prosthesis is held within the slot, the anchoring sites of the prosthesis are exposed.

24. The kit as claimed in claim 23, in which the holding device includes an additional elongate holding component spanning the gap between the free ends of the bowed end face of the C-shaped holder, the elongate holding component having a face defining a slot adapted to accommodate the straight portion of a D-shaped prosthesis when the bowed portion thereof is accommodated in the slot of the holder, so that anchoring sites along the straight portion of the D-shaped prosthesis are exposed.
